(19) United States
(12) Patent Application Publication
(10) Pub. No.: US 2008/0021274 A1
(43) Pub. Date: Jan. 24, 2008

(54) ENDOSCOPIC MEDICAL DEVICE WITH LOCKING MECHANISM AND METHOD

(75) Inventors: Lex Bayer, Palo Alto, CA (US); Diel Mark, Menlo Park, CA (US)

Correspondence Address: SQUIRE, SANDERS & DEMPSEY L.L.P., 1 MARITIME PLAZA, SUITE 300, SAN FRANCISCO, CA, 94111, US

(73) Assignee: Avantis Medical Systems, Inc., Sunnyvale, CA (US)

(21) Appl. No.: 11/751,605

(22) Filed: May 21, 2007

Related U.S. Application Data
(63) Continuation-in-part of application No. 11/673,470, filed on Feb. 9, 2007.
Application No. 11/673,470 is a continuation-in-part of application No. 11/672,020, filed on Feb. 6, 2007.
Application No. 11/673,470 is a continuation-in-part of application No. 11/626,189, filed on Jan. 23, 2007.
Application No. 11/673,470 is a continuation-in-part of application No. 11/609,838, filed on Dec. 12, 2006.
Application No. 11/673,470 is a continuation-in-part of application No. 11/215,660, filed on Aug. 29, 2005.
Application No. 11/673,470 is a continuation-in-part of application No. 11/030,559, filed on Jan. 5, 2005, now abandoned.
(60) Provisional application No. 60/801,923, filed on May 19, 2006.

(51) Int. Cl. A61B 1/04

(52) U.S. Cl. 600/112; 600/117; 600/181

(57) ABSTRACT

An endoscopic system having an endoscope carrying an endoscopic medical device that can be an auxiliary rear-ward facing camera or medical tool for cutting, ablation, cutting, suturing, retracting, manipulating, suction, or irrigation. A locking assembly allows the user of the system to lock the rotational orientation of a distal portion of endoscopic medical device within a distal end of the endoscope. The locking assembly has a locking device attached to the distal portion of the endoscopic medical device and a cap that is permanently or removably attached to the distal end of the endoscope. When desired, the user of the system engages the locking device and the cap together to prevent rotation. The locking device can include one more flexible members that can contract when pushed through a working channel of the endoscope and expand to engage mating features on the cap. Magnets can also be used to prevent rotation.

### TECHNICAL FIELD OF THE INVENTION

The present invention generally relates to an endoscopic medical device and, more particularly, to an endoscopic medical device with a locking mechanism.

### BACKGROUND OF THE INVENTION

A conventional endoscope is a medical device comprising a flexible tube, and a camera and a light source mounted on the distal end of the flexible tube. The endoscope is insertable into an internal body cavity through a body orifice to examine the body cavity and tissues for diagnosis. The tube of the endoscope has one or more longitudinal channels, through which an instrument can reach the body cavity to take samples of suspicious tissues or to perform other surgical procedures such as polypectomy.

There are many types of endoscopes, and they are named in relation to the organs or areas with which they are used. For example, gastroscopes are used for examination and treatment of the esophagus, stomach and duodenum; colonoscopes for the colon; bronchoscopes for the bronchi; laparoscopes for the peritoneal cavity; sigmoidoscopes for the rectum and the sigmoid colon; arthroscopes for joints; cystoscopes for the urinary bladder; and angioscopes for the examination of blood vessels.

Each endoscope has a single forward viewing camera mounted at the distal end of the flexible tube to transmit an image to an eyepiece or video camera at the proximal end. The camera is used to assist a medical professional in advancing the endoscope into a body cavity and looking for abnormalities. The camera provides the medical professional with a two-dimensional view from the distal end of the endoscope. To capture an image from a different angle or in a different portion, the endoscope must be repositioned or moved back and forth. Repositioning and movement of the endoscope prolongs the procedure and causes added discomfort, complications, and risks to the patient. Additionally, in an environment similar to the lower gastrointestinal tract, flexures, tissue folds and unusual geometries of the organ may prevent the endoscope's camera from viewing all areas of the organ. The unseen area may cause a potentially malignant (cancerous) polyp to be missed.

This problem can be overcome by providing an auxiliary camera and an auxiliary light source. The auxiliary camera and light source can be oriented to face the main camera and light source, thus providing an image of areas not viewable by the endoscope's main camera. This arrangement of cameras and light sources can provide both front and rear views of an area or an abnormality. In the case of polypectomy where a polyp is excised by placing a wire loop around the base of the polyp, the camera arrangement allows better placement of the wire loop to minimize damage to the adjacent healthy tissue.

Since the main camera and light source face the auxiliary camera and light source, the main light source interferes with the auxiliary camera, and the auxiliary light source interferes with the main camera. Light interference is the result of the light from a light source being projected directly onto the lens of a camera. This may cause light glare, camera blooming, or over saturation of light, resulting in inferior image quality.

Additionally, because of space constraint, the auxiliary camera and auxiliary light source are typically smaller than the main camera and main light source and use different technologies. Different types of cameras often require different levels of illumination. For example, the main camera generally requires a higher level of illumination and needs a more powerful light source. As a result, the auxiliary camera is often exposed to a significant amount of glare caused by the powerful main light source. The glare can be reduced with the use of polarizing filters which must have a particular orientation.

The use of multiple endoscopic medical devices is often necessary or desirable during a surgical or other procedure. For example, an endoscope can be used for viewing and an endoscopic medical device can be inserted into one of the working channels of the endoscope. The endoscopic medical device can be another endoscope, an auxiliary camera, or other device. Examples of other endoscopic medical devices include without limitation cutting, ablation, grasping, snaring, retracting, manipulating, suturing, suction, and irrigation tools. It is often necessary to fix the rotational position of the inserted endoscopic device to prevent it from moving longitudinally or rotationally from its desired position or orientation.

Accordingly, there is a need for a mechanism for selectively locking the rotational orientation of a polarizing filter of an auxiliary camera and a polarizing filter attached at the distal end of an endoscope. There is also a need for a locking mechanism that allows for locking of the relative rotational orientation of an endoscope and other types of endoscopic medical devices inserted into the endoscope. The present invention satisfies these and other needs.

### SUMMARY OF THE INVENTION

In accordance with an aspect of the invention, an endoscopic medical device comprises n endoscopic medical device comprises a tubular body comprising a central longitudinal axis, an inner locking device attached to the tubular body and comprising an inner locking member that is radially movable relative to the tubular body, and an outer locking device having an opening sized to allow the tubular body to pass through the outer locking device, the outer locking device comprising an outer locking member sized to engage the inner locking member such that the tubular body is prevented from rotating about the longitudinal axis relative to the outer locking device.

The endoscopic medical device in other aspects of the invention further comprises an imaging device, a light source, and a polarizing filter at a distal end of the tubular body, wherein the polarizing filter is disposed over either one or both of the imaging device and the light source.

In other aspects of the invention, the outer locking device comprises a side wall extending longitudinally, a slot formed through the side wall, and an end wall attached to the side wall at an angle, wherein the side wall extends proximally from the end wall and the opening of the outer locking device is formed through the end wall. In further aspects, the end wall has a hole for an imaging device and a hole for a light source, and the outer locking device further comprises a polarizing filter disposed over either one or both of the hole for an imaging device and the hole for a light source.

In yet other aspects of the invention, the outer locking device comprises a cylindrical wall extending longitudinally, and the cylindrical wall is concentric with the opening. The outer locking device in further aspects comprises at least one rib disposed on an inner surface of the cylindrical wall. The cylindrical wall in further aspects has at least one notch formed on an inner surface of the cylindrical wall.

In other aspects of the invention, the inner locking member includes a flexible flap comprising a first end attached to the tubular body and a second end that is radially movable relative to the tubular body, and the outer locking member is sized to engage the second end of the flap. In yet other aspects, the inner locking member is a wire spring comprising a medial portion and at least one end attached to the tubular body, the medial portion being radially movable relative to the tubular body, and the outer locking member is sized to engage the medial portion of the wire spring. In further aspects, the medial portion of the wire spring and the outer locking member are longitudinally oriented. In other further aspects, the medial portion of the wire spring is disposed further away from the longitudinal axis than the rest of the wire spring. In still other further aspects, the inner locking device further comprises a second wire spring comprising a medial portion and at least one end attached to the tubular body, the medial portion of the second wire spring being radially movable relative to the tubular body, the outer locking device further comprises a second inner locking member that is sized to engage the medial portion of the second wire spring.

The endoscopic medical device in other aspects of the invention comprises a position indicator attached to the tubular body, the position indicator being longitudinally aligned with the inner locking member. In further aspects, the position indicator comprises a distal portion, a proximal portion, and at least one end attached to the tubular body, the distal portion oriented at a first acute angle relative to the longitudinal axis, the proximal portion oriented at a second acute angle relative to the longitudinal axis. In other further aspects, the first acute angle is less than the second acute angle.

In other aspects, the medial portion of the wire spring comprises a curved segment that has a peak disposed further away from the longitudinal axis than the rest of the wire spring, and the outer locking member is sized to engage the peak. In yet other aspects, the medial portion of the wire spring comprises a distal segment oriented at a first acute angle to the longitudinal axis, a proximal segment oriented at a second acute angle to the longitudinal axis, and an intermediate segment disposed between the distal segment and the proximal segment, and the outer locking member is sized to engage the intermediate segment.

In still other aspects of the invention, the inner locking member is a leaf spring comprising at least one end attached to the tubular body, the leaf spring being radially movable relative to the tubular body. In further aspects, the leaf spring comprises a distal portion oriented at a first angle to the longitudinal axis, a proximal portion oriented at a second angle to the longitudinal axis, and an intermediate section oriented longitudinally and disposed between the distal portion and the proximal portion, and the outer locking member is longitudinally oriented to engage the intermediate section. In other further aspects, the inner locking device further comprises a second leaf spring comprising at least one end attached to the tubular body, the second leaf spring being radially movable relative to the tubular body, and the outer locking device further comprises a second outer locking member that is sized to engage the second leaf spring.

In yet other aspects of the invention, the inner locking member is a torsion spring disposed circumferentially around a portion of the tubular body, the torsion spring comprising a first portion attached to the tubular body, a second portion that is radially movable relative to the tubular body, and a locking element on the second portion, and the outer locking member is sized to engage the locking element. In further aspects, the torsion spring is formed from a sheet material that bends around the tubular body, and the locking element is a rib on the movable portion of the torsion spring. In other further aspects, the torsion spring is formed from a wire that bends around the tubular body, the wire comprising a locking segment, and the locking element is the locking segment of the wire. In detailed aspects, the wire of the inner locking device further comprises a second locking segment, and the outer locking device comprises a second outer locking member sized to engage the second locking segment of the wire. In yet other aspects, the inner locking device includes a plurality of torsion springs formed from separate wires, each wire bent around the tubular body and comprising a locking segment, and the outer locking device includes a plurality of outer locking members, each of the outer locking members sized to engage at least one of the locking segments.

In accordance with other aspects of the invention, an endoscopic system comprises an endoscope having a longitudinal channel, an endoscopic medical device including a tubular body, wherein the endoscopic medical device is disposed in the longitudinal channel, and a locking assembly that interlocks the endoscope and the endoscopic medical device to prevent relative rotational movement between the endoscope and the endoscopic medical device.

In accordance with yet other aspects of the invention, a method of configuring an endoscopic system comprises inserting a tubular body of an endoscopic medical device into a channel of an endoscope, wherein an inner locking device is attached to the tubular body, and wherein when the tubular body is inside the channel the inner surface of the channel radially compresses the inner locking device, and continuing to insert the tubular body of the endoscopic medical device until the inner locking device comes out of the channel, wherein the inner locking device extends radially outwards to engage an outer locking device attached to the endoscope, and wherein the engagement prevents the tubular body from rotating relative to the endoscope.

The features and advantages of the invention will be more readily understood from the following detailed description which should be read in conjunction with the accompanying drawings.

### BRIEF DESCRIPTION OF THE DRAWINGS

FIG. 1 shows a perspective view of an endoscope with an imaging assembly inserted into the endoscope according to one embodiment of the present invention.

FIG. 2 shows a perspective view of the distal end of an insertion tube of the endoscope of FIG. 1 with a polarizer cap.

FIG. 3 shows a perspective back view of the polarizer cap of FIG. 2.

FIG. 4 shows a perspective view of the imaging assembly shown in FIG. 1.

FIG. 5 shows a perspective view of the distal ends of the endoscope and imaging assembly of FIG. 1 with a cross-sectional view of the lens barrel of the imaging assembly.

FIG. 6 is a perspective view of an imaging system showing an endoscope and an endoscopic medical device inserted through a working channel of the endoscope, and showing a locking assembly attached to the endoscope and the endoscopic medical device, the locking assembly having an inner locking device and an outer locking device.

FIG. 7 is a front, perspective view of the of the outer locking device of FIG. 6 shown detached from the endoscope, the outer locking device is shown with flexible finger-like elements for securing the outer locking device to the endoscope and an opening with mating features adapted to engage the inner locking device.

FIG. 8 is a rear, perspective view of the outer locking device of FIG. 6 shown detached from the endoscope, the outer locking device is shown with a polarizing filter covering a cutout adjacent the opening with mating features.

FIG. 9 is a perspective view of the distal end region and functional portion of the endoscopic medical device of FIG. 6 shown without the endoscope, the distal end region is shown attached to the inner locking, which is in the form of a flap, and the functional portion is shown with a camera oriented backwards.

FIG. 10 is a perspective view of the locking assembly of FIG. 6 showing the flap of FIG. 9 engaged with mating features in the opening of the outer locking device, the outer locking device is shown attached to the distal end of an endoscope.

FIG. 11 is a perspective view of a locking assembly showing magnets on an outer locking device.

FIG. 12 is a perspective view of an inner locking device showing three wires attached to a distal end region of an endoscopic medical device, the three wires for preventing rotation of the distal end region relative to a distal end of an endoscope, two of the wires each having a curved segment allowing for ease of insertion into an opening of an outer locking device.

FIG. 13 is a perspective view of an inner locking device showing three wires attached to a distal end region of an endoscopic medical device, the three wires for preventing rotation of the distal end region relative to a distal end of an endoscope, two of the wires each having a stepped segment allowing for tactile feedback during insertion into an opening of an outer locking device.

FIG. 14 is a perspective view of the inner locking device of FIG. 13 showing the stepped segment engaged with mating features formed in an opening of an outer locking device so as to prevent rotation of the distal end region.

FIG. 15 is a perspective view of an inner locking device showing a torsion spring formed of a sheet material, the torsion spring shown with a plurality of longitudinally oriented ribs with edges for engaging mating features of an outer locking device.

FIG. 16 is a cross-sectional view of the inner locking device of FIG. 15 showing a first portion and a second portion of the torsion spring, the first portion is shown fixedly attached to and extending tangentially away from a distal end region of an endoscopic medical device, the second portion is shown disposed at a distance from the distal end region and being radially movable toward or away from the distal end region.

FIG. 17 is a perspective view of an inner locking device showing a wire torsion spring shown detached from an endoscopic medical device, the wire torsion spring having three coiled portions configured to coil around a tubular body of the endoscopic medical device and two longitudinally oriented portions configured to be movable relative to the tubular body.

FIG. 18 is a perspective view of an inner locking device showing a wire torsion spring shown detached from an endoscopic medical device, the wire torsion spring having two coiled portions configured to coil around a tubular body of the endoscopic medical device and two longitudinally oriented portions configured to be movable relative to the tubular body.

FIG. 19 is a perspective view of an inner locking device showing two wire torsion springs shown attached to a tubular body of an endoscopic medical device, each wire torsion spring having two coiled portions configured to coil around a tubular body of the endoscopic medical device and one longitudinally oriented portion configured to be movable relative to the tubular body, the first and second wires extending tangentially away from the tubular body at opposing directions.

FIG. 20 is an end view of the inner locking device of FIG. 19 showing the longitudinally oriented portions of the two wire torsion springs engaged with mating features formed in an opening of an outer locking device.

FIG. 21 is a perspective view of an inner locking device showing two leaf springs attached to a tubular body of an endoscopic medical device, the leaf springs each shown with segments oriented at an angle from the tubular body and a longitudinally oriented intermediate segment.

FIG. 22 is a perspective view of an outer locking device showing mating features in the form of slots adjacent an opening sized to allow passage of the tubular body of an endoscopic medical device, the slots sized to engage the intermediate segment of the inner locking device of FIG. 21 to prevent rotation of the tubular body relative to the outer locking device.

### DETAILED DESCRIPTION OF EMBODIMENTS OF THE INVENTION

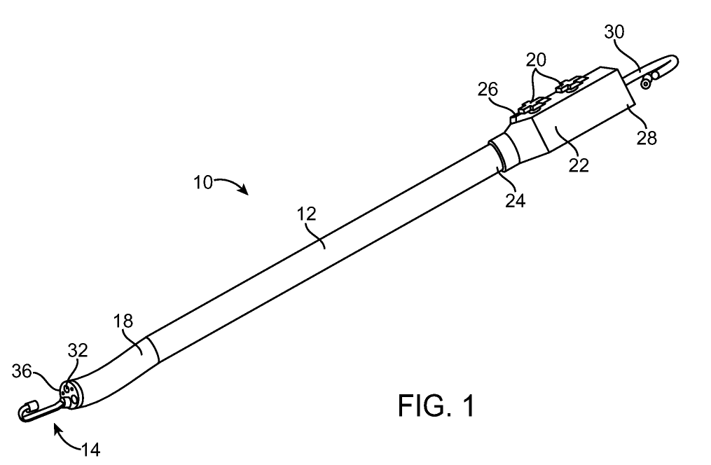

Referring now in more detail to the exemplary drawings, wherein like reference numerals designate corresponding or like elements among the several views, there is shown in FIG. 1 a first exemplary endoscope 10 of the present invention. This endoscope 10 can be used in a variety of medical procedures in which imaging of a body tissue, organ, cavity or lumen is required. The types of procedures include, for example, anoscopy, arthroscopy, bronchoscopy, colonoscopy, cystoscopy, EGD, laparoscopy, and sigmoidoscopy.

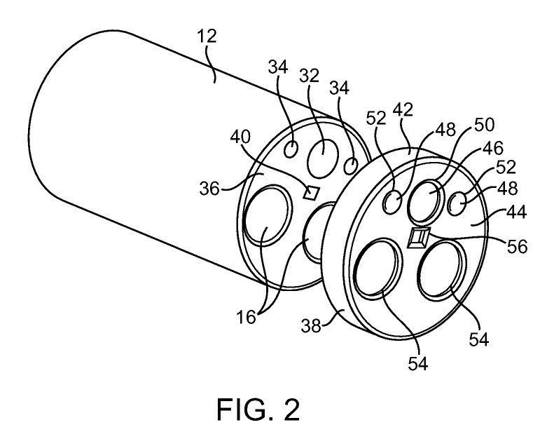

The endoscope 10 of FIG. 1 includes an insertion tube 12 and an imaging assembly 14, a section of which is housed inside the insertion tube 12. As shown in FIG. 2, the insertion tube 12 has two longitudinal channels 16. In general, however, the insertion tube 12 may have any number of longitudinal channels. An instrument can reach the body cavity to perform any desired procedures, such as to take samples of suspicious tissues or to perform other surgical procedures such as polypectomy. The instruments may be, for example, a retractable needle for drug injection, hydraulically actuated scissors, clamps, grasping tools, electrocoagulation systems, ultrasound transducers, electrical sensors, heating elements, laser mechanisms and other ablation means. In some embodiments, one of the channels can be used to supply a washing liquid such as water for washing. Another or the same channel may be used to supply a gas, such as CO₂ or air into the organ. The channels 16 may also be used to extract fluids or inject fluids, such as a drug in a liquid carrier, into the body. Various biopsy, drug delivery, and other diagnostic and therapeutic devices may also be inserted via the channels 16 to perform specific functions.

The insertion tube 12 preferably is steerable or has a steerable distal end region 18 as shown in FIG. 1. The length of the distal end region 18 may be any suitable fraction of the length of the insertion tube 12, such as one half, one third, one fourth, one sixth, one tenth, or one twentieth. The insertion tube 12 may have control cables (not shown) for the manipulation of the insertion tube 12. Preferably, the control cables are symmetrically positioned within the insertion tube 12 and extend along the length of the insertion tube 12. The control cables may be anchored at or near the distal end 36 of the insertion tube 12. Each of the control cables may be a Bowden cable, which includes a wire contained in a flexible overlying hollow tube. The wires of the Bowden cables are attached to controls 20 in the handle 22. Using the controls 20, the wires can be pulled to bend the distal end region 18 of the insertion tube 12 in a given direction. The Bowden cables can be used to articulate the distal end region 18 of the insertion tube 12 in different directions.

As shown in FIG. 1, the endoscope 10 may include a control handle 22 connected to the proximal end 24 of the insertion tube 12. Preferably, the control handle 22 has one or more ports and/or valves (not shown) for controlling access to the channels 16 of the insertion tube 12. The ports and/or valves can be air or water valves, suction valves, instrumentation ports, and suction/instrumentation ports. As shown in FIG. 1, the control handle 22 may additionally include buttons 26 for taking pictures with an imaging device on the insertion tube 12, the imaging assembly 14, or both.

The proximal end 28 of the control handle 22 may include an accessory outlet 30 (FIG. 1) that provides fluid communication between the air, water and suction channels and the pumps and related accessories. The same outlet 30 or a different outlet can be used for electrical lines to light and imaging components at the distal end of the endoscope 10.

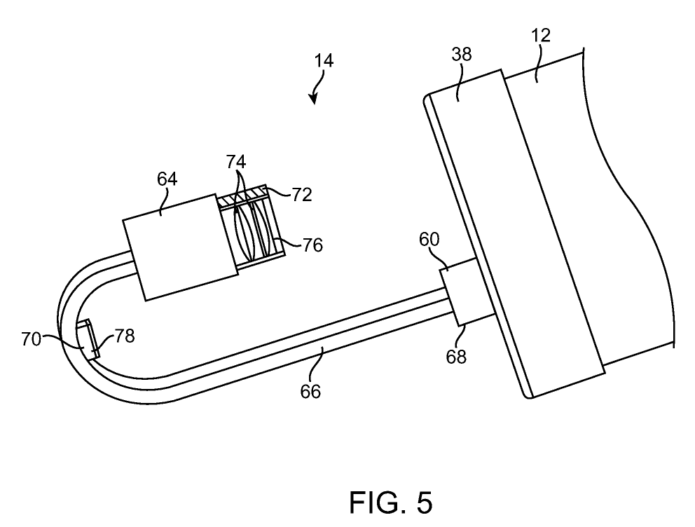

As shown in FIG. 2, the endoscope 10 also includes a main imaging device 32 and main light sources 34, both of which are disposed at the distal end 36 of the insertion tube 12, and a polarizer cap 38 that is adapted to be mounted on the distal end 36 of the insertion tube 12 to cover the main imaging device 32 and main light sources 34. FIG. 2 shows the polarizer cap 38 removed from the distal end 36 of the insertion tube 12, and FIG. 5 shows the polarizer cap 38 mounted on the distal end 36 of the insertion tube 12.

The main imaging device 32 at the distal end 36 of the insertion tube 12 may include, for example, a lens, single chip sensor, multiple chip sensor or fiber optic implemented devices. The main imaging device 32, in electrical communication with a processor and/or monitor, may provide still images or recorded or live video images. The light sources 34 may be light emitting diodes (LEDs) or fiber optical delivery of light from an external light source. The light sources 34 preferably are equidistant from the main imaging device 32 to provide even illumination. The intensity of each light source 34 can be adjusted to achieve optimum imaging. The circuits for the main imaging device 32 and light sources 34 may be incorporated into a printed circuit board (PCB). As shown in FIG. 2, the insertion tube 12 has a channel 40 for supplying a liquid such as water for cleaning the lenses of the main imaging device 32 and the light sources 34.

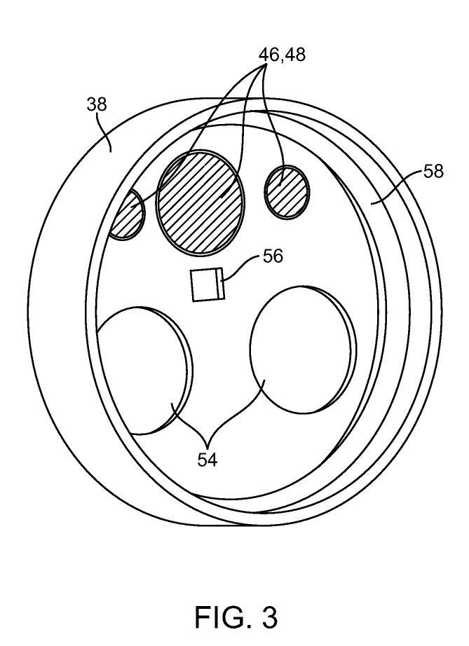

The polarizer cap 38, as shown in FIGS. 2 and 3, includes a cylindrical sidewall 42, an end wall 44, and polarizing filters 46, 48 mounted on the end wall 44. The cylindrical sidewall 42 and end wall 44 may form an integral part that is made by injection molding of a suitable biocompatible material such as medical grade plastics. The end wall 44 preferably has an opening 50 for accommodating the polarizing filter 46 for the main imaging device 32. The opening 50 may have any arrangement suitable for retaining the polarizing filter 46. For example, the opening 50 may have a recessed lip for receiving the polarizing filter 46. The polarizing filter 46 can be placed in the recessed lip and fixed there by adhesive bonding or by a mechanical snap fit.

The end wall 44 preferably has an opening 52 for accommodating the polarizing filter 48 for each of the light sources 34. The opening 52 may have any arrangement suitable for retaining the polarizing filter 48. One example of such suitable arrangement is the recessed lip described above. The end wall 44 preferably has an opening 54 for each of the instrument channels 16 so that the cap 38 does not block the channels 16.

The end wall 44 may further include an opening 56 for the channel 40 for supplying a liquid to clean the lenses of the imaging device 32 and the light sources 34. Preferably, the cap 38 has one or more features that allow liquid from the channel 40 to reach over the cap 38 to clean the exterior surfaces of polarizing filters 46, 48. For example, the end wall 44 of the cap 38 may be sufficiently thin to allow the liquid from the channel 40 to reach over the cap 38 to clean the exterior surfaces of polarizing filters 46, 48. Alternatively, the cap 38 may have variable thickness and/or angled features that allow liquid from the channel 40 to reach the polarizing filters 46, 48. Furthermore, the cap 38 may have a ramp, plate or channel that allows liquid from the channel 40 to reach the polarizing filters 46, 48. The locations, configurations and sizes of the openings 50, 52, 54, 56 preferably correspond to the locations, configurations and sizes of the main imaging device 32, light sources 34, channels 16, and clean liquid channel 40, respectively.

As shown in FIG. 3, the cap 38 preferably has a ring 58 located around the inner perimeter of the cap 38. The ring 58 helps secure the cap 38 to the distal end region of the insertion tube 12. In a preferred embodiment, the ring 58 is made from a compressive material such as silicon. Alternatively, the ring 58 can be made from other compressive materials, such as compressive rubbers, polymers and/or foams. The ring 58 may be attached the inner perimeter of the cap 38 by any suitable means such as adhesive bonding, mechanical over molding, or plastic snap features.

The inside diameter of the ring 58 preferably is slightly smaller than the outer diameter of the insertion tube 12 so that the ring 58 can apply a compressive force to the outer surface of the insertion tube 12. This compressive force preferably is sufficient to create the necessary friction force to ensure that the cap 38 remains in the same position and orientation during a medical procedure, yet to allow the cap 38 to be slide on and off of the insertion tube 12 without difficulty.

Alternatively, the cap 38 may have any other type of arrangement for attachment to the insertion tube 12. For example, the cap 38 may have clasps which snap on to the insertion tube 12. In some embodiments, the attachment may be similar to the way in which a suction cap for endoscopic mucosal resection is attached to a colonoscope, as is well known in the art.

The terms “polarizing filter” and “polarizer” as used in this specification refer to any device that blocks one or more components of light while allowing one or more other components to pass through. In some cases, polarizing filters may be made from a material that blocks light waves traveling in all planes from passing through the filter except for light waves propagating in one specific plane of orientation, often referred to as the plane of polarization or the plane of transmission. Polarizing filters may be constructed using various techniques that use light absorption, reflection, scattering or birefringence to block light from passing through the filter that is not orientated parallel with the plane of transmission.

When one polarizing filter is placed in front of another polarizing filter and non-coherent natural white light is passed through the two polarizing filters, the amount of light that passes through the two polarizing filters is proportional to the relative angle of orientation of the two filters. This is because when the polarization plane of the two filters is at the same angle of orientation, the majority of light waves in the plane of transmission will pass through both filters. As one of the filters is rotated, light that is polarized by the first filter is then attenuated or blocked by the second filter. The maximum amount of light reduction or extinction occurs when the polarizing planes of the two filters are orientated at 90° relative to each other. It is common to find polarizing filters that when orientated at 90° provide 99% or greater extinction of light transmission.

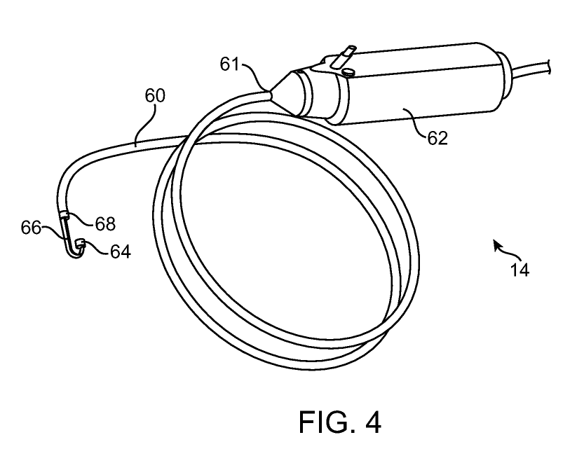

As shown in FIGS. 4 and 5, the imaging assembly 14 may include a tubular body 60, a handle 62 connected to the proximal end 61 of the tubular body 60, an auxiliary imaging device 64, a link 66 that provides physical and/or electrical connection between the auxiliary imaging device 64 to the distal end 68 of the tubular body 60, and an auxiliary light source 70 (FIG. 5).

As shown in FIG. 5, the imaging assembly 14 is used to provide an auxiliary imaging device at the distal end of the endoscope 10. To this end, the imaging assembly 14 is placed inside one of the channels 16 of the endoscope's insertion tube 12 with its auxiliary imaging device 64 disposed beyond the distal end 36 of the insertion tube 12. This can be accomplished by first inserting the distal end of the imaging assembly 14 into the insertion tube's channel 16 from the endoscope's handle 18 and then pushing the imaging assembly 14 further into the assembly 14 until the auxiliary imaging device 64 and link 66 of the imaging assembly 14 are positioned outside the distal end 36 of the insertion tube 12 as shown in FIG. 5.

As shown in FIG. 5, the auxiliary imaging device 64 may include a lens barrel 72 having one or more lenses 74, an imaging sensor, and a printed circuit board (PCB). The imaging sensor may be an electronic device which converts light incident on photosensitive semiconductor elements into electrical signals. The imaging sensor may detect either color or black-and-white images. The signals from the imaging sensor can be digitized and used to reproduce an image that is incident on the imaging sensor. Two commonly used types of image sensors are Charge Coupled Devices (CCD) such as a VCC-5774 produced by Sanyo of Osaka, Japan and Complementary Metal Oxide Semiconductor (CMOS) camera chips such as an OVT 6910 produced by OmniVision of Sunnyvale, Calif.

The endoscope 10 preferably includes a polarizing filter 76 placed in front of the auxiliary imaging device 64. The polarizing filter 76 may be placed inside the lens barrel 72. Alternatively, the polarizing filter 76 may be placed directly onto the image sensor itself, or incorporated at various other locations in the lens barrel 72 such as at the end closest to the imaging sensor, or even between the lenses 74. Furthermore, the polarizing filter 76 may be simply placed in front of the auxiliary imaging device.

When the imaging assembly 14 is properly installed in the insertion tube 12, the auxiliary imaging device 64 of the imaging assembly 14 preferably faces backwards towards the main imaging device 32 as illustrated in FIG. 3. The auxiliary imaging device 64 may be oriented so that the auxiliary imaging device 64 and the main imaging device 32 have adjacent or overlapping viewing areas. Alternatively, the auxiliary imaging device 64 may be oriented so that the auxiliary imaging device 64 and the main imaging device 32 simultaneously provide different views of the same area. Preferably, the auxiliary imaging device 64 provides a retrograde view of the area, while the main imaging device 32 provides a front view of the area. However, the auxiliary imaging device 64 could be oriented in other directions to provide other views, including views that are substantially parallel to the axis of the main imaging device 32.

As shown in FIGS. 2 and 3, the link 66 connects the auxiliary imaging device 64 to the distal end 68 of the tubular body 60. Preferably, the link 66 is a flexible link that is at least partially made from a flexible shape memory material that substantially tends to return to its original shape after deformation. Shape memory materials are well known and include shape memory alloys and shape memory polymers. A suitable flexible shape memory material is a shape memory alloy such as nitinol. The flexible link 66 is straightened to allow the distal end of the imaging assembly 14 to be inserted into the proximal end of assembly 14 of the insertion tube 12 and then pushed towards the distal end 36 of the insertion tube 12. When the auxiliary imaging device 64 and flexible link 66 are pushed sufficiently out of the distal end 36 of the insertion tube 12, the flexible link 66 resumes its natural bent configuration as shown in FIG. 3. The natural configuration of the flexible link 66 is the configuration of the flexible link 66 when the flexible link 66 is not subject to any force or stress. When the flexible link 66 resumes its natural bent configuration, the auxiliary imaging device 64 faces substantially back towards the distal end 36 of the insertion tube 12 as shown in FIG. 5.

In the illustrated embodiment, the auxiliary light source 70 (as well as other components) of the imaging assembly 14 is placed on the flexible link 66, in particular on the curved concave portion of the flexible link 66. The auxiliary light source 70 provides illumination for the auxiliary imaging device 64 and may face substantially the same direction as the auxiliary imaging device 64 as shown in FIG. 5.

The endoscope 10 includes another polarizing filter 78 placed in front of the auxiliary light source 70. The polarizing filter 78 may be attached to the auxiliary light source 70 by any suitable means such as adhesive bonding or welding.

The flexible link 66 may be encapsulated or shrouded by flexible tubing, heat-shrinkable tubing, urethanes, rubber or silicon so as to allow smooth profile transition from the tubular body 60 to the imaging device 64. This encapsulation may be translucent to allow light from the light source 70 to project through the encapsulation, or the encapsulation may include a window section around the light source 70.

Since the main imaging device 32 and its light source 34 face the auxiliary imaging device 64 and its light source 70, the light sources 34, 45 of the imaging devices 32, 64 may interfere with the opposing imaging device 64, 32. That is, the main light source 34 may shine directly into auxiliary imaging device 64 and the auxiliary light source 70 may shine directly into the main imaging device 32, degrading both images.

To eliminate or reduce the light interference, the polarization plane of the polarizing filter 46 for the main imaging device 32 may be set at a substantially 90° angle from the polarization plane of the polarizing filter 78 for the auxiliary light source 70. With this arrangement, the light, which is emitted from the auxiliary light source 70 and passes though the polarizing filter 78, may be filtered out by the polarizing filter 46 and may not reach the main imaging device 32. Additionally or alternatively, the polarization plane of the polarizing filter 76 for the auxiliary imaging device 64 may be set at a substantially 90° angle from the polarization plane of the polarizing filters 48 for the main light sources 34. With this arrangement, the light, which is emitted from the main light sources 34 and passes though the polarizing filters 48, may be filtered out by the polarizing filter 76 and may not reach the auxiliary imaging device 64.

Moreover, to provide illumination, the polarization plane of the polarizing filter 46 for the main imaging device 32 may be substantially aligned with the polarization plane of the polarizing filters 48 for the main light sources 34 so that the light, which is emitted from the main light sources 34 and passes though the polarizing filters 48, may pass through the polarizing filter 46 and may be received by the main imaging device 32. Additionally or alternatively, the polarization plane of the polarizing filter 76 for the auxiliary imaging device 64 may be substantially aligned with the polarization plane of the polarizing filter 78 for the auxiliary light source 70 so that the light, which is emitted from the auxiliary light source 70 and passes though the polarizing filter 78, may pass through the polarizing filter 76 and may be received by the auxiliary imaging device 64.

The desired relative orientations of the polarizing filters' the polarization planes, as set forth above, may be achieved in any suitable manner. For example, the polarization planes of the polarizing filters 46, 48 for the main imaging device 32 and main light sources 34 may be aligned and fixed in the polarizer cap 38, and the polarization planes of the polarizing filters 76, 78 for the auxiliary imaging device 64 and auxiliary light source 70 may be aligned and fixed in the imaging assembly 14. Then the imaging assembly 14 may be rotated within the channel 16 of the insertion tube 12 by means of its handle 62 until the polarization planes of the polarizing filters 76,78 in the imaging assembly 14 are at a substantially 90° angle from the polarization planes of the polarizing filters 46, 48 in the polarizer cap 38.

The orientations of the polarizing filters' the polarization planes may be determined and set during attachment by viewing a light with a known polarization passing through polarizing filters. Alternatively, the polarizing filters may have asymmetrical shapes or other locating features so that the orientations of their polarization planes may be readily determined.

The auxiliary imaging device 64 and its light source 70 may be connected to a control box (not shown) via electrical conductors that extend from the imaging device 64 and light source 70; through the link 66, tubular body 60, and handle 62; to the control box. The electrical conductors may carry power and control commands to the auxiliary imaging device 64 and its light source 70 and image signals from the auxiliary imaging device 64 to the control box.

The control box includes at least an image and signal processing device and a housing in which the image and signal processing device is disposed, although the control box can be configured in any suitable manner. The housing may include a control panel and connectors. The control panel includes buttons and knobs for controlling the functionalities of the control box.

The image and signal processing device may include one or more integrated circuits and memory devices along with associated discrete components. The device allows image signals from the imaging devices 32, 64 to be processed for the enhancement of image quality, extraction of still images from the image signals, and conversion of video format for compatibility with the display device.

The control box preferably processes the video image signal from the auxiliary imaging device 64 and transmits it to a display device such as a television or a monitor such as a liquid crystal display monitor. Still images can be captured from the video image signal. The video image or still image may be displayed on the display device. The display device may also include textual data that are used to display information such as patient information, reference numbers, date, and/or time.

The image signal from the main imaging device 32 may also be processed by the control box in the same way that the image signal from the auxiliary imaging device 64 is processed. The images from the main and auxiliary imaging devices 32, 64 may be displayed on two separate monitors or on the same monitor with a split screen.

The control box may further be used to adjust the parameters of the imaging devices 32, 64 and their light sources 34, 70, such as brightness, exposure time and mode settings. The adjustment can be done by writing digital commands to specific registers controlling the parameters. The registers can be addressed by their unique addresses, and digital commands can be read from and written to the registers to change the various parameters. The control box can change the register values by transmitting data commands to the registers.

The control box may additionally be used as an interface to the patient records database. A large number of medical facilities now make use of electronic medical records. During the procedure relevant video and image data may need to be recorded in the patient electronic medical records (EMR) file. The signal processing circuit can convert image and video data to a format suitable for filing in the patient EMR file such as images in jpeg, tif, or .bmp format among others. The processed signal can be transmitted to the medical professional's computer or the medical facilities server via a cable or dedicated wireless link. A switch on the control panel can be used to enable this transmission. Alternatively the data can be stored with a unique identification for the patient in electronic memory provided in the control box itself. The signal processing circuit can be utilized to convert the video and image data to be compatible with the electronic medical records system used by the medical professional. The processing may include compression of the data. A cable or a wireless link may be used to transmit the data to a computer.

During endoscopy, a technician may first install the polarizer cap 38 onto the endoscope's insertion tube 12. A physician may then insert the endoscope into a body cavity through an orifice of the body. Once the endoscope is inserted, the physician may decide to use the imaging assembly 14 in order to obtain a rear-viewing image of a certain tissue. The physician may straighten the flexible link 66 of the imaging assembly 14 and insert the straightened distal end of the imaging assembly 14 into the channel 16 of the endoscope's insertion tube 12 from the handle 22. The imaging assembly 14 can then be pushed towards the distal end 36 of the insertion tube 12. When the auxiliary imaging device 64 and flexible link 66 are pushed out of the distal end 36 of the insertion tube 12, the flexible link 66 resumes its natural bent configuration as shown in FIG. 2. The main imaging device 32 now captures a front-viewing image, and the auxiliary imaging device 64 simultaneously captures a rear-viewing image of the same area. The physician may then rotate the imaging assembly 14 so that the polarization planes of the polarizing filters 76,78 in the imaging assembly 14 are at a substantially 90° angle from the polarization planes of the polarizing filters 46, 48 in the polarizer cap 38. Once the correct orientation has been established, the physician locks or fixes the orientation of the imaging assembly 14 relative to the insertion tube 12. The physician then continues with the procedure.

The above-described embodiment is merely one of many alternative embodiments of the present invention. In one other alternate embodiment, polarizing filters are placed over only the auxiliary imaging device 64 of the imaging assembly 14 and the main light sources 34 to reduce light interference between them. In this embodiment, a low intensity auxiliary light source 70 may be used for the auxiliary imaging device 64 to alleviate any bright spots that could be seen by the main imaging device 32. This arrangement allows maximum light intake by the main imaging device 32 without light loss caused by a polarizing filter. Similarly, in another alternative embodiment, polarizing filters are placed over only the main imaging device 32 and the auxiliary imaging device 64. These two embodiments are useful depending on the types of imaging sensors used in the endoscope, specifically their light sensitivities, resistance to blooming, and dynamic ranges, as well as depending on the types of light sources used in the endoscope and their illumination intensities and/or wave length spectrums.

In an alternate embodiment, the orientation features include a feature, such as a pin, rod or geometric feature, affixed to and protruding slightly away from the imaging assembly, and a feature, such as a cup and tube, on the polarizer cap that mates with the corresponding feature on the imaging assembly. The features may be made from a compressive material such as rubber so that when the two features are engaged a substantial force is needed to break the engagement. In this manner, the physician would first slide the imaging assembly past the distal end of the insertion tube and then, under the guidance of the auxiliary imaging device, rotate the imaging assembly to achieve the correct relative orientation between the polarizing filters. When the correct relative orientation between the polarizing filters is achieved, the physician may retract the imaging assembly so that the two features engage and lock together. To later disengage the features, the physician may forcefully advance the imaging assembly.

In yet a further alternate embodiment, the distal end of the imaging assembly includes a mechanism that can fix the position of the imaging assembly in the channel of the insertion tube. Such a mechanism may include the use of inflatable balloons, springs that are actuated via guide-wires, mechanical engagement arrangements, or frictional methods such as large diameter compressive regions incorporating rubber or foam.

In the present application, the terms “insertion tube,” “imaging assembly” and “endoscope” are interchangeable, may have the same or similar meanings, and may have the same or similar features and functions. Different terms are used in the application for ease of identification and description. Additionally, such a description should not be used to limit the breadth of the application. The use of “insertion tube,” “imaging assembly”, or “endoscope” merely refers to possible types of instruments in the broad field of endoscopy and the invention may be applied to many forms of endoscopes and medical imaging devices.

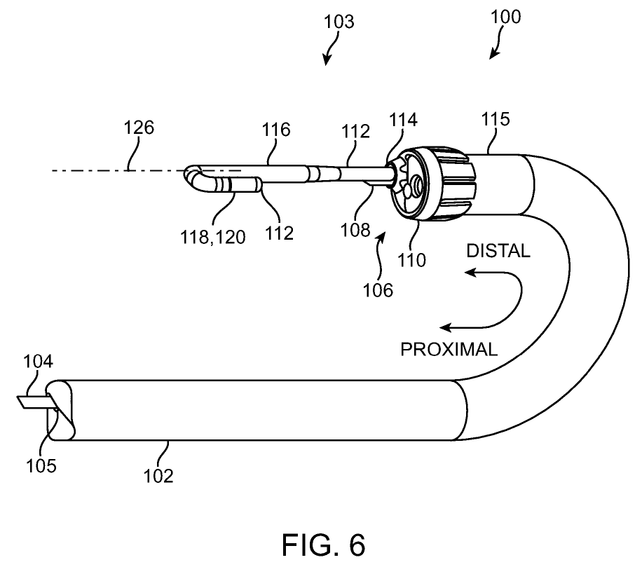

Referring now to FIG. 6, there is shown an endoscopic system 100 in accordance with an embodiment of the present invention. The system comprises an endoscope 102 and an endoscopic medical device 103 having a flexible tubular body 104 inserted through a working channel 105 of the endoscope 102. The system 100 also comprises a locking assembly 106 that includes an inner locking device 108 and a cap or outer locking device 110. The distal end region 112 of the tubular body 104 is shown extending through an opening 114 formed in the outer locking device 110. The inner locking device 108 is fixedly attached to the distal end region 112 of the endoscopic medical device 103. The outer locking device 110 is fixedly attached to the distal end 115 of the endoscope 102. In some embodiments, the outer locking device 110 is permanently attached to the endoscope, while in other embodiments it is removably attached to allow for cleaning and maintenance.

In the illustrated embodiment of FIG. 6, the endoscopic medical device 103 is a retrograde-viewing apparatus having a functional device 116 that comprises a camera or imaging device 118, a light source 120, and a polarizing filter 122.

Other types of endoscopic medical devices known in the art may be used in other embodiments. Examples of other types of endoscopic medical devices include without limitation catheters, prosthesis delivery instruments, as well as cutting, ablation, grasping, snaring, retracting, manipulating, suturing, suction, and irrigation tools. As such, the functional device 116 in other embodiments may comprise a cutting blade, electrode, or fluid carrying tube as appropriate for the type of endoscopic medical device.

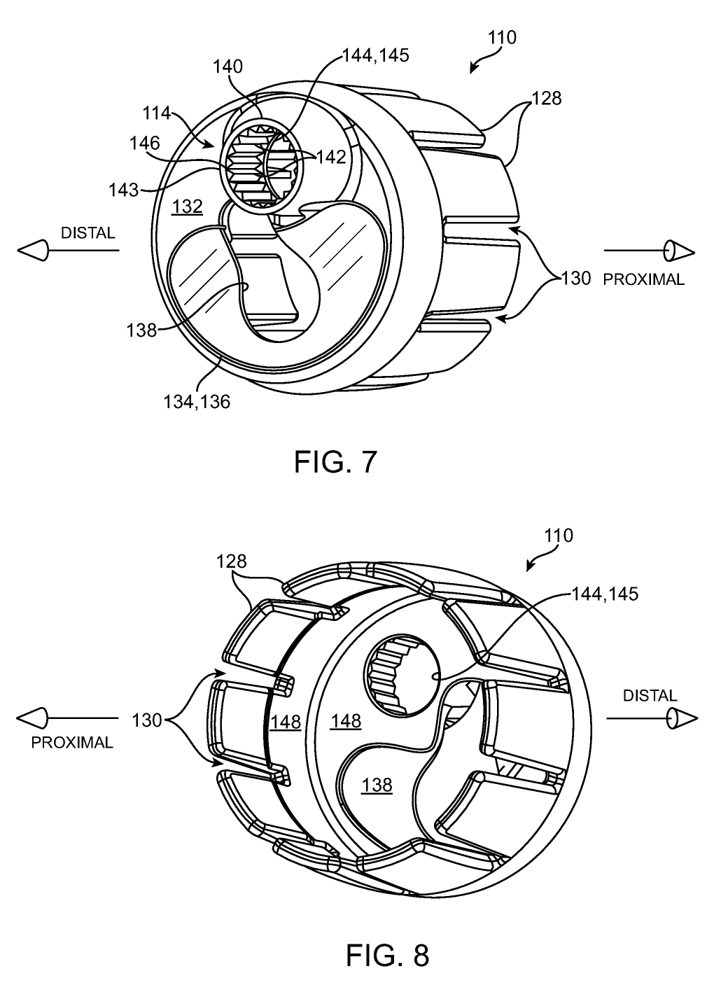

In FIG. 7, the outer locking device 110 of FIG. 6 is shown detached from the endoscope 102. In use, the functional device 116 of endoscopic medical device 103 may be rotated when desired about a central longitudinal axis 26 (FIG. 6) of the distal end region 112. When the functional device 116 is at its desired orientation or position, the user may then cause the outer locking device and the inner locking device to engage each other by sliding the endoscopic medical device 103 within the endoscope 102. The outer locking device 110 comprises a plurality of outer locking members that are sized to engage an inner locking member of the inner locking device 108 such that the functional device 116 adjacent the distal end region 112 is prevented from rotating relative to the outer locking device 110.

The outer locking device 110 comprises a side wall 128 extending longitudinally, a plurality of slots 130 formed through the side wall 128, and an end wall 132 attached to the side wall 128. The end wall 132 is substantially perpendicular to the side wall 128. The side wall 128 also extends proximally from the end wall 132. The end wall 132 also has a hole 134 for an imaging device of the endoscope 102, a hole 136 for the light source or sources on the endoscope 102, and a hole for an air/water channel of the endoscope 102. In the illustrated embodiment, the holes for the endoscope imaging device, light source, and air/water channel are combined as one opening or cutout. The outer locking device 110 also has a polarizing filter 138 that is disposed over the area of the cutout corresponding to the light sources on the endoscope 102. The area of the cutout corresponding to the imaging device on the endoscope 102 is not covered by the polarizing filter 138.

In other embodiments, the polarizing filter 138 only covers the imaging device on the endoscope 102. In yet other embodiments, the polarizing filter 138 covers both the imaging device and light source on the endoscope 102. In still other embodiments, the various holes for the endoscope's imaging device, light source, and air/water channel are separate or located in other areas of the cap 110 according to the model or type of endoscope the cap is intended to be used with.

Referring again to FIG. 7, the outer locking device 110 further comprises a cylindrical wall 140 that extends longitudinally and distally from the end wall 132. The cylindrical wall 140 is concentrically aligned with the opening 114 for the tubular body 104 of the endoscopic medical device 103. The plurality of outer locking members is disposed within the cylindrical wall 140. In the illustrated embodiment, the outer locking members are in the form of teeth or longitudinally oriented ribs that extend from the distal edge 143 of the opening 114 to a step 144 at the proximal edge of the opening 114. The step 144 may function as a stop to prevent the inner locking device 108 from being pulled inadvertently past the outer locking device 110. Also, there are longitudinally oriented notches or grooves 146 formed into the cylindrical wall 140 between the ribs 146. It will be appreciated by persons skilled in the art that mating features apart from ribs and grooves may be used on the outer locking device 110 to engage the inner locking device 108. Examples of other mating features include without limitation pins, slots, and springs.

In FIG. 7, the opening 114 is a through hole with a circumference that is entirely surrounded by the cylindrical wall 140 of the outer locking device 110. In other embodiments, the opening 114 may be configured differently. For example, the opening 114 may be a slot, rather than a hole, formed in the outer locking device 110. The slot in other embodiments may be semi-circular in shape and may only be partially surrounded by a wall 140. Outer locking members or mating may be formed at or adjacent the slot in other embodiments so as to engage an inner locking device 108.

Referring next to FIG. 8, the end wall 132 of the outer locking device 110 has a proximally facing surface 148 surrounding by the side wall 128. The proximally facing surface 148 can have a recessed portion and a raised portion which allows the end wall 132 to mate with the surface geometry found on the heads of endoscope 102s. The side wall 128 and slots 130 form flexible finger-like members that may snap onto the distal end of the endoscope 102 to secure the outer locking device 110 in position and prevent the locking device from detaching from the endoscope 102.

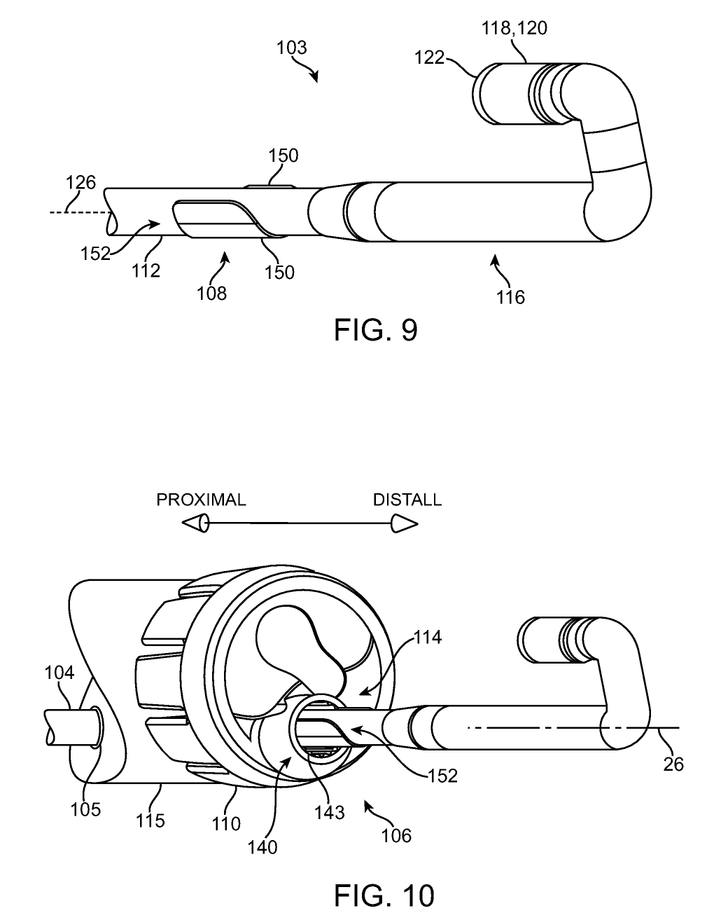

In FIG. 9, the distal portion of the endoscopic medical device 103 of FIG. 6 is shown in detail. The inner locking device 108 has an inner locking member 149 in the form of a flexible flap. The flap comprises a first end 150 attached to the distal end region 112 and a second end 152 that is radially movable relative to the distal end region 112. The flap can be made of a flexible material such as metal, Mylar, or other sheet material. The flap can be attached to the distal end region 112 with adhesives.

When the endoscopic medical device 103 is slid into an endoscope 102, compression forces from sides of the working channel 105 of the endoscope 102 keep the flap furled against the distal end region 112. When the flap reaches the opening of the cap 110, it unfurls and the movable end 152 of the flap engages the grooves 146 or ribs 142 on the cylindrical wall 140, as shown in FIG. 10. When the endoscopic medical device 103 has an imaging device, as is illustrated, this locking feature keeps the imaging device rotationally steady with respect to the distal end 115 of endoscope 102. The flap 108 includes a chamfer or radius at the corners of its movable end to ensure that when the endoscopic medical device 103 is retracted or inserted through the working channel 105 and the cap 110, the flap can furls or unfurls as appropriate. In other embodiments, a plurality of flaps can be used to better prevent rotation.

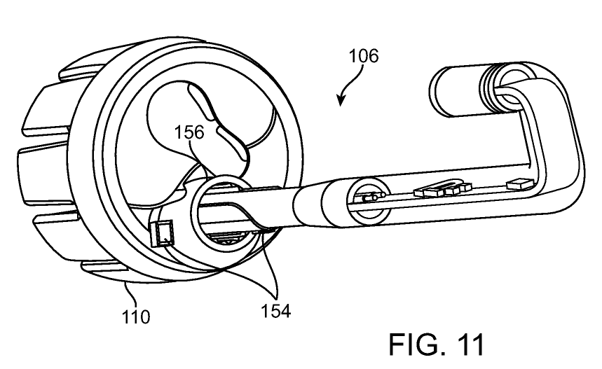

Referring now to FIG. 11, a pair of magnets 154 are attached to the end wall 132 of the cap 110 adjacent the opening. An inner and outer ring 156 made of non-magnetic material is disposed around the distal end region 112 of the endoscopic medical device 103 and holds projections 158 made of a metal or other material that magnetically interact with the magnets 154. The rings are attached to the distal end region 112 with adhesives, or other means known in the art, such that the rotational orientation of the endoscopic medical device 103 is secured as appropriate. In other embodiments, the magnets 154 are attached to the distal end region 112 and the projections 158 are attached to the cap 110.

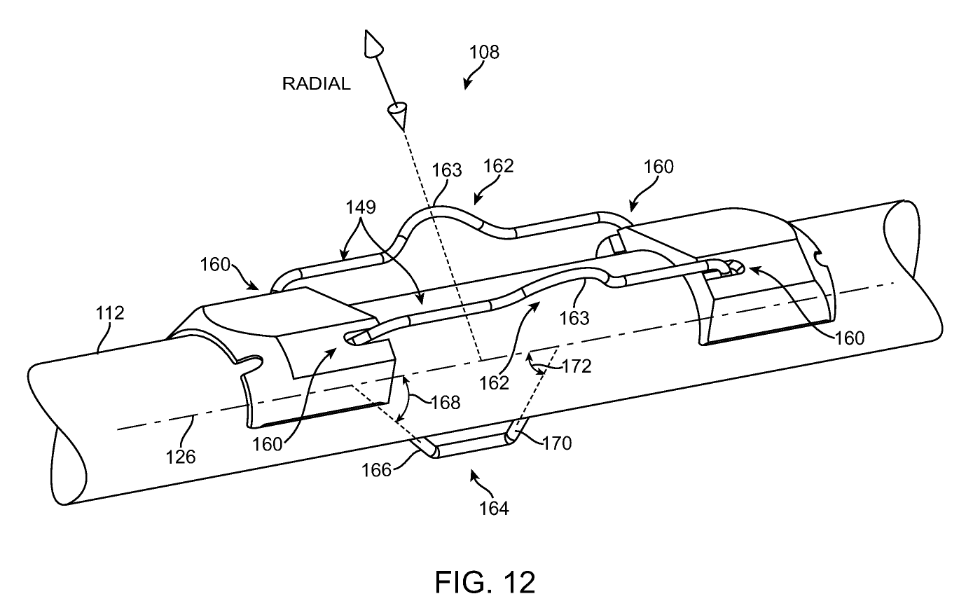

FIG. 12 shows an alternative embodiment of an inner locking device 108 having two inner locking members 149 in the form a wire springs, each of which are attached to and extending away from the distal end region 112 of the tubular body 104 of the endoscopic medical device 103. Each spring comprises two opposite ends 160 that are attached to the distal end region 112 and a medial or intermediate segment 162 disposed between the two opposite ends. The intermediate segments each include a curved portion that has a peak 163 that is preferably disposed further away from the longitudinal axis 26 than the rest of the wire springs. The curve portion is configured to allow for ease of insertion of the springs through the opening of the outer locking device 110.

Each wire spring 108 is mounted such that the intermediate segment 162 is radially movable relative to the distal end region 112. Preferably, the ends are loosely connected loosely the distal end region 112 by two slotted mounting blocks firmly attached to the distal end region 112. The loose connections allows each spring 108 to stretch or contract more readily, as appropriate, when its intermediate segment 162 is moved radially away or toward the distal end region 112. The ribs or notches 146 at the cylindrical wall 140 of outer locking device 110 are sized to engage the intermediate segments to prevent a functional device 116 of an endoscopic medical device 103 from rotating relative to the outer locking device 110.

The endoscopic medical device 103 optionally includes a position indicator 164 attached to the distal end region 112. The position indicator 164 may be a wire that is preferably longitudinally aligned with the inner locking members 149. That is, the position indicator 164 and the inner locking members 149 are located at substantially the same longitudinal position along the distal end region 112. The position indicator 164 includes ends which are attached to the distal end region 112. The position indicator 164 also includes a distal portion 166 oriented at a first acute angle 168 and a proximal portion 170 oriented at a second acute angle 172 relative to the longitudinal axis 26. Preferably, the first acute 168 angle is less than the second acute angle 172. In this way, the distal end region 112 may be easily slid in a forward or distal direction through the opening of the outer locking device 110.

In use, the position indicator 164 and the inner locking members 149 on the inner locking device 108 are pushed forward or distally past the distal edge 143 of the opening 114 of the outer locking device 110, such that the inner locking members 149 are not engaged with the outer locking device 110. The rotational position of the functional device 116 may then be adjusted as desired. When the desire rotational position is achieved, the endoscopic medical device 103 is pull backward or proximally until the inner locking members 149 slide into engagement with the outer locking device 110. The position indicator 164 provides tactile feedback to the person pulling the endoscopic medical device 103. When the proximal portion 170 hits the distal edge 143 (FIG. 7) of the opening 114, the person will feel increased resistance to pulling, which indicates that the endoscopic medical device 103 only needs to be pulled a little further to lock its rotational orientation. Additional resistance to pulling is provided by the step 144 (FIGS. 7 and 8) to indicate that the inner and outer locking devices 108, 110 are fully engaged and that the endoscopic medical device is locked against rotation.

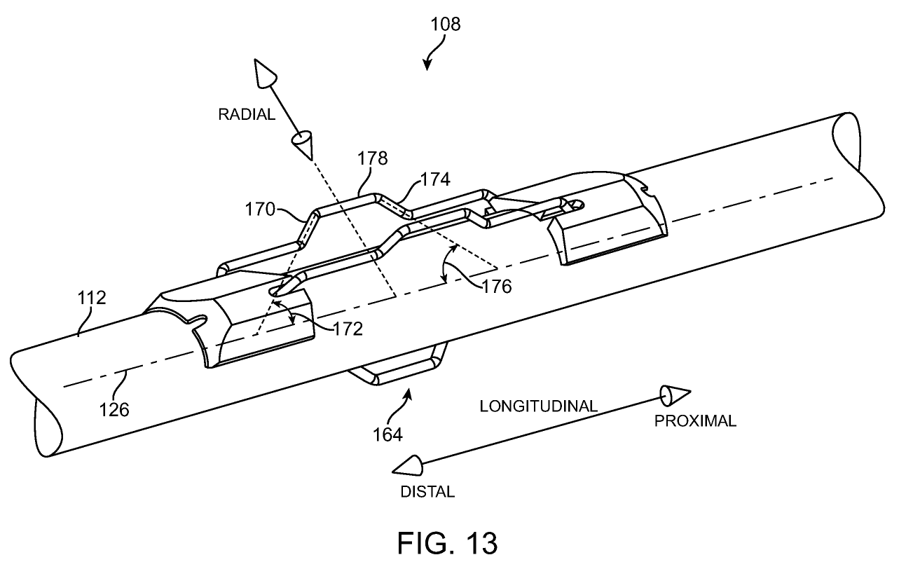

In FIG. 13 there is shown an embodiment of an inner locking device 108. Here, the two inner locking members 149 in the form a longitudinally oriented wire springs, each of which are attached to and extending away from the distal end region 112 of the tubular body 104 of the endoscopic medical device 103. The spring comprises two opposite ends that are attached to the distal end region 112 and a medial portion 162 disposed between the two opposite ends. The medial portion 162 includes a distal segment 170 oriented at a first acute angle 172 to the longitudinal axis 26, a proximal segment 174 oriented at a second acute angle 176 to the longitudinal axis 26, an intermediate segment 178 disposed between the distal and proximal segments. The first and second acute angles 172, 176 may be selected to provide additional tactile feedback to indicate that rotational position is locked.

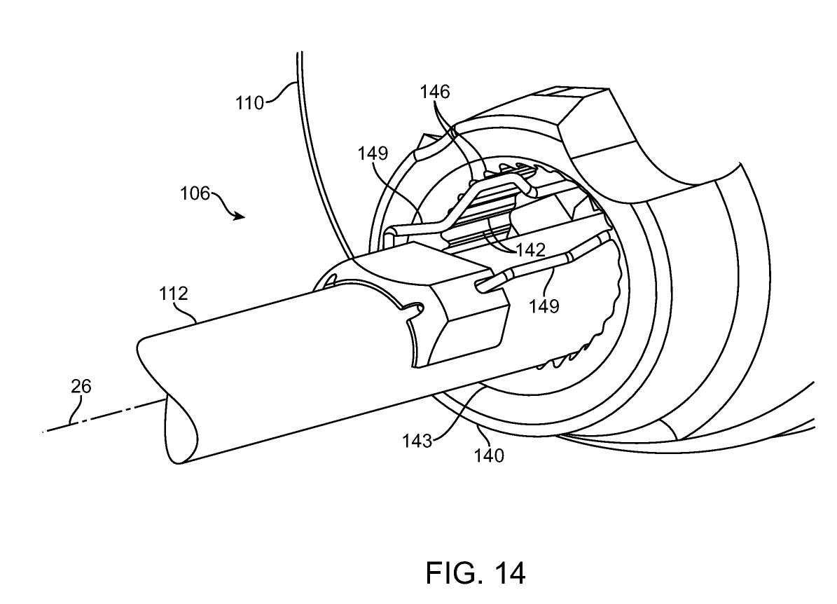

Referring now to FIG. 14, the inner locking device 108 of FIG. 13 is shown engaged with an outer locking device 110. The intermediate segment 178 has moved or flexed radially toward the distal end region 112. The ribs or grooves of the cylindrical wall 140 are sized to engage the intermediate segment 178 to prevent a functional device adjacent the distal end region 112 from rotating about the longitudinal axis 26 relative to the outer locking device 110. In this way, the desired orientation of any polarizing filter on the functional device is maintained. With the outer locking device 110 securely attached to the distal end of an endoscope, the functional device would also be prevented from rotating relative to the distal end of the endoscope.

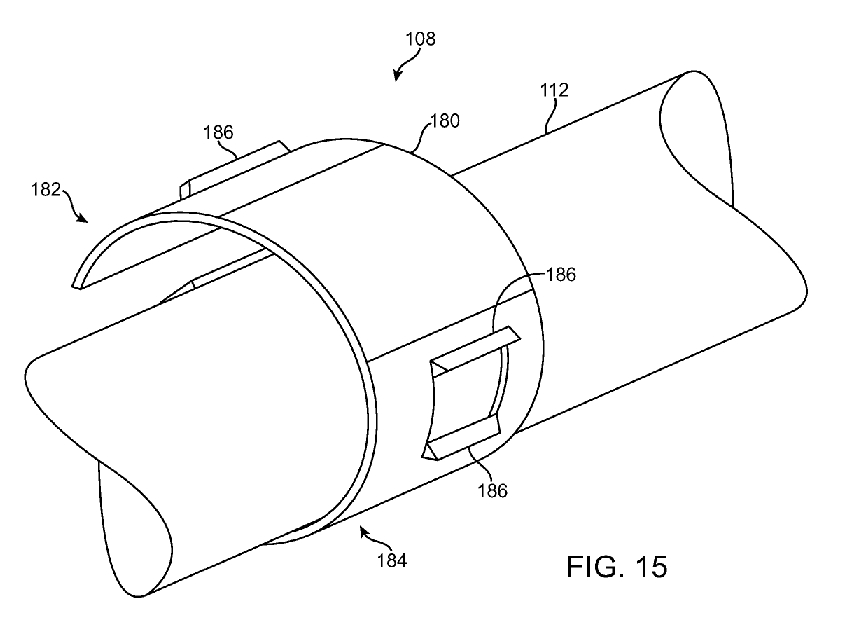

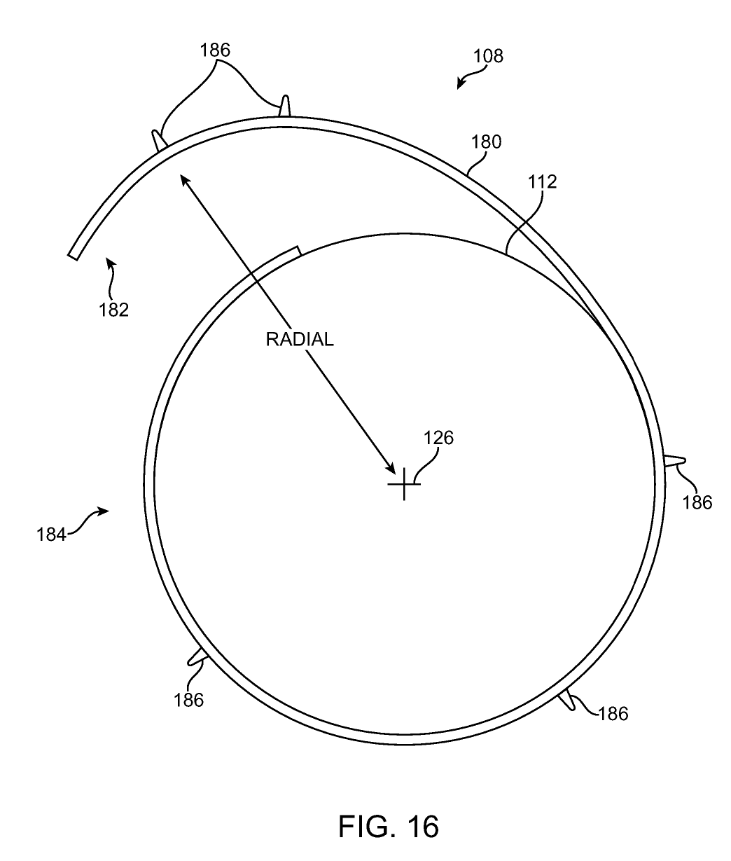

In FIGS. 15 and 16 there is shown another embodiment of an inner locking device 108. The inner locking device 108 has an inner locking member 149 in the form of a torsion spring 180. The torsion spring 180 is disposed circumferentially around a portion of a distal end region 112 of a tubular body of an endoscopic medical device. The torsion spring 180 includes a first portion 182 fixedly attached to the distal end region 112, a second portion 184 that is radially movable relative to the distal end region 112, and a plurality of locking elements 186 on the first portion 182 and the second portion 184. Preferably, the torsion spring 180 is formed from a sheet material that bends around the distal end region 112. The locking elements 186 are in the form of ribs protruding radially outward from the torsion spring outer surface. The grooves or ribs of an outer locking device 110 are sized to engage the locking elements to prevent rotation of a functional device adjacent the distal end region 112.

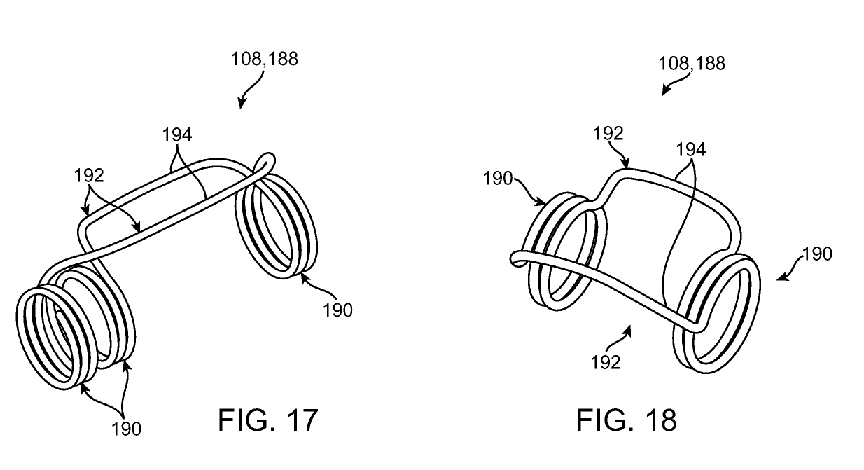

Referring next to FIGS. 17-20, the inner locking device 108 can have inner locking members 149 in the form of torsion springs 194, 204 formed of wire. These torsion springs, like the torsion spring formed of a sheet material shown in FIGS. 15 and 16, extend tangentially away from a curved surface of the distal end region 112. The wire torsion springs 194, 204 comprise a first segment 190, 200 attached to the distal end region 112 and a second segment 192, 202 that is radially movable relative to the distal end region 112.

In FIGS. 17 and 18, an inner locking device 108 comprises one torsion wire spring 188, which is shown without an endoscopic medical device 103 for clarity of illustration. In use, the torsion wire spring 188 is attached to a distal end region 112 of an endoscopic medical device 103. The torsion spring comprises first portions 190 that are attached to the distal end region 112 and second portions 192 that are radially movable relative to the distal end region 112. The first portions bend around the distal end region 112. A longitudinally oriented segment 194 of the second portion 192 functions as a locking element that engages the outer locking device 110 during use.

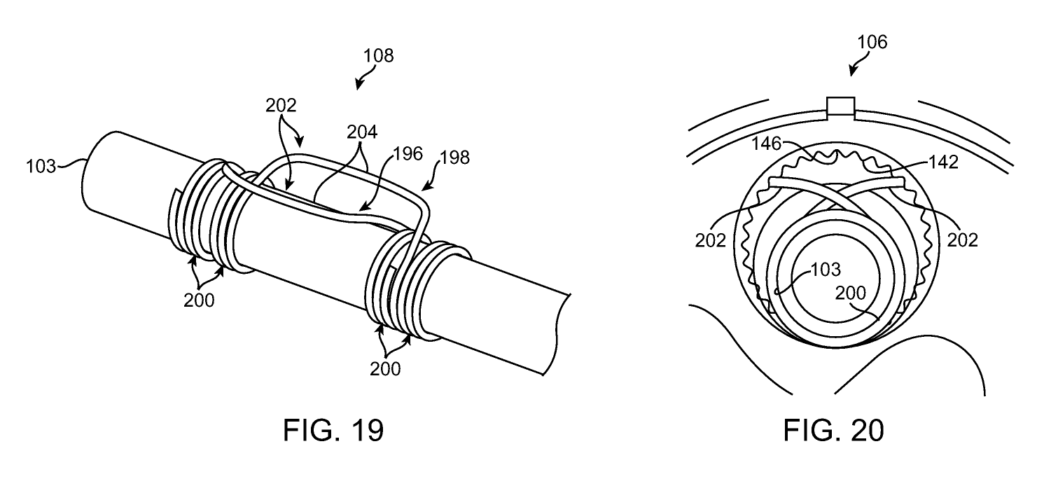

In FIGS. 19 and 20, an inner locking device 108 comprises two separate torsion wire springs 196, 198, which are shown mounted onto a portion of an endoscopic medical device 103. The wire springs 196, 198 comprise first portions 200 that are attached to the distal end region 112 and second portions 202 that are radially movable relative to the endoscopic medical device 103. The first portions 200 bend around the endoscopic medical device 103. A longitudinally oriented segment 204 of each second portion 202 functions as a locking element that engages the outer locking device 110, as shown in FIG. 20.

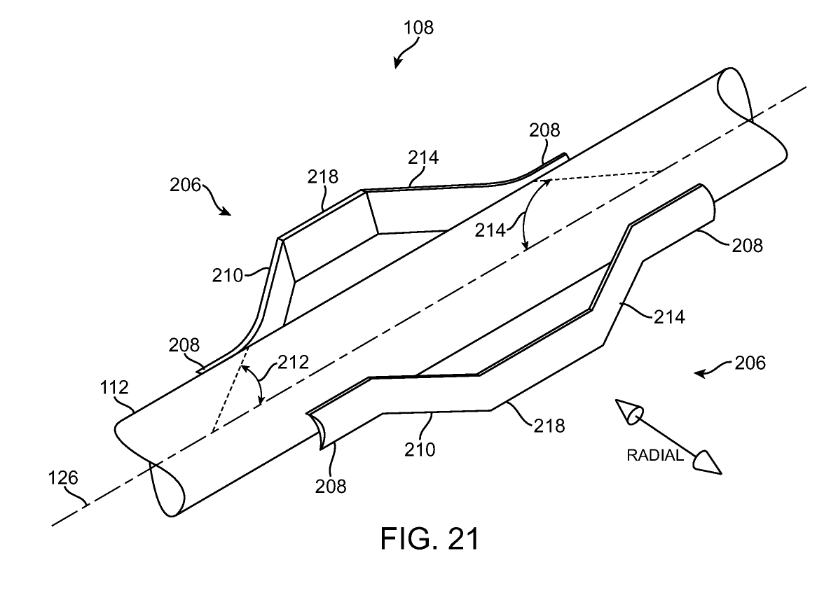

Referring to FIG. 21, an inner locking device 108 in other embodiments can include a plurality of leaf springs 206 each comprising one or two ends 208 which are attached to a distal end region 112 of an endoscopic medical device 103. The leaf springs 206 are radially movable relative to the distal end region 112. The leaf springs 206 comprise a distal portion 210 oriented at a first acute angle 212 to the central longitudinal axis 26 of the distal end region 112 and a proximal portion 214 oriented at a second acute angle 216 to the longitudinal axis 26. The acute angles 212, 214 may be the same of different as appropriate to create a desired tactile feedback. The leaf springs 206 also include an intermediate section 218 oriented longitudinally and disposed between the distal and proximal portions 210, 214. In use, an outer locking member of the outer locking device engages the intermediate section to prevent a functional device attached the distal end region 112 from rotating relative to an outer locking device 110 when it is desired that the inner and outer locking devices be locked together against rotation.

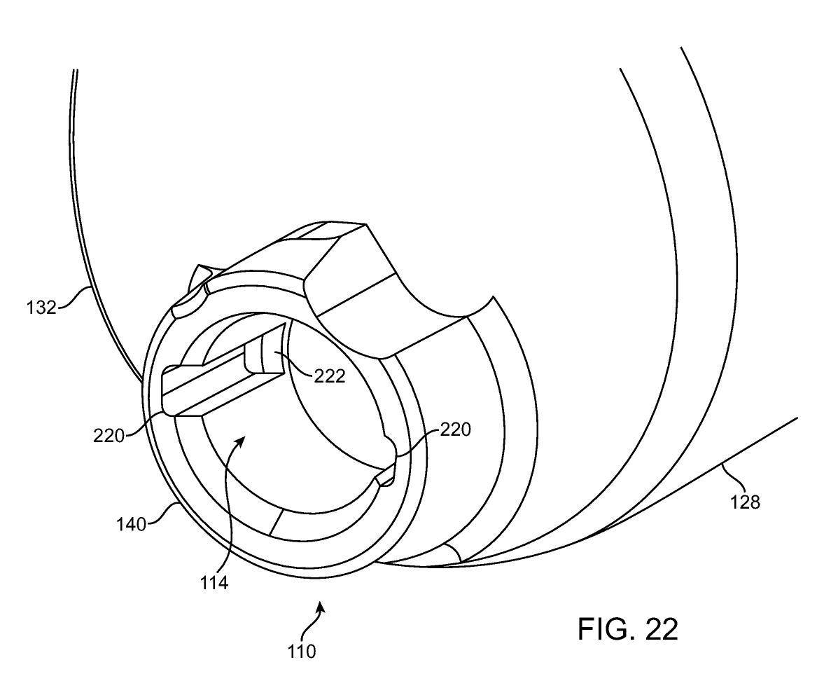

In FIG. 22, another embodiment of an outer locking device 110 is shown. A pair of slots or notches 220 is formed in a cylindrical wall 140. The notches 220 are sized to engage the intermediate sections 218 of the leaf springs 206 of FIG. 21. The outer locking device 110 also includes a step 222 located at the distal end of the notches 220. The step 222 may function as a stop to prevent the inner locking device 108 from being pulled inadvertently past an outer locking device 110.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

1. An endoscopic medical device comprising:
a tubular body comprising a central longitudinal axis;
an inner locking device attached to the tubular body and comprising an inner locking member that is radially movable relative to the tubular body; and
an outer locking device having an opening sized to allow the tubular body to pass through the outer locking device, the outer locking device comprising an outer locking member sized to engage the inner locking member such that the tubular body is prevented from rotating about the longitudinal axis relative to the outer locking device.

2. The endoscopic medical device of claim 1, further comprising an imaging device, a light source, and a polarizing filter at a distal end of the tubular body, wherein the polarizing filter is disposed over either one or both of the imaging device and the light source.

3. The endoscopic medical device of claim 1, wherein the outer locking device comprises a side wall extending longitudinally, a slot formed through the side wall, and an end wall attached to the side wall at an angle, wherein the side wall extends proximally from the end wall and the opening of the outer locking device is formed through the end wall.

4. The endoscopic medical device of claim 3, wherein:
the end wall has a hole for an imaging device and a hole for a light source; and
the outer locking device further comprises a polarizing filter disposed over either one or both of the hole for an imaging device and the hole for a light source.

5. The endoscopic medical device of claim 1, wherein:
the outer locking device comprises a cylindrical wall extending longitudinally; and
the cylindrical wall is concentric with the opening.

6. The endoscopic medical device of claim 5, wherein the outer locking device comprises at least one rib disposed on an inner surface of the cylindrical wall.

7. The endoscopic medical device of claim 5, wherein the cylindrical wall has at least one notch formed on an inner surface of the cylindrical wall.

8. The endoscopic medical device of claim 1, wherein:
the inner locking member includes a flexible flap comprising a first end attached to the tubular body and a second end that is radially movable relative to the tubular body; and
the outer locking member is sized to engage the second end of the flap.

9. The endoscopic medical device of claim 1, wherein:
the inner locking member is a wire spring comprising a medial portion and at least one end attached to the tubular body, the medial portion being radially movable relative to the tubular body; and
the outer locking member is sized to engage the medial portion of the wire spring.

10. The endoscopic medical device of claim 9, wherein the medial portion of the wire spring and the outer locking member are longitudinally oriented.

11. The endoscopic medical device of claim 9, wherein the medial portion of the wire spring is disposed further away from the longitudinal axis than the rest of the wire spring.

12. The endoscopic medical device of claim 9, wherein:
the inner locking device further comprises a second wire spring comprising a medial portion and at least one end attached to the tubular body, the medial portion of the second wire spring being radially movable relative to the tubular body; and
the outer locking device further comprises a second inner locking member that is sized to engage the medial portion of the second wire spring.

13. The endoscopic medical device of claim 9, further comprising a position indicator attached to the tubular body, the position indicator being longitudinally aligned with the inner locking member.

14. The endoscopic medical device of claim 13, wherein the position indicator comprises a distal portion, a proximal portion, and at least one end attached to the tubular body, the distal portion oriented at a first acute angle relative to the longitudinal axis, the proximal portion oriented at a second acute angle relative to the longitudinal axis.

15. The endoscopic medical device of claim 14, wherein the first acute angle is less than the second acute angle.

16. The endoscopic medical device of claim 9, wherein:
the medial portion of the wire spring comprises a curved segment that has a peak disposed further away from the longitudinal axis than the rest of the wire spring; and
the outer locking member is sized to engage the peak.

17. The endoscopic medical device of claim 9, wherein:
the medial portion of the wire spring comprises a distal segment oriented at a first acute angle to the longitudinal axis, a proximal segment oriented at a second acute angle to the longitudinal axis, and an intermediate segment disposed between the distal segment and the proximal segment; and
the outer locking member is sized to engage the intermediate segment.

18. The endoscopic medical device of claim 1, wherein:
the inner locking member is a leaf spring comprising at least one end attached to the tubular body, the leaf spring being radially movable relative to the tubular body.

19. The endoscopic medical device of claim 18, wherein:
the leaf spring comprises a distal portion oriented at a first angle to the longitudinal axis, a proximal portion oriented at a second angle to the longitudinal axis, and an intermediate section oriented longitudinally and disposed between the distal portion and the proximal portion; and
the outer locking member is longitudinally oriented to engage the intermediate section.

20. The endoscopic medical device of claim 18, wherein the inner locking device further comprises a second leaf spring comprising at least one end attached to the tubular body, the second leaf spring being radially movable relative to the tubular body; and
the outer locking device further comprises a second outer locking member that is sized to engage the second leaf spring.

21. The endoscopic medical device of claim 1, wherein:
the inner locking member is a torsion spring disposed circumferentially around a portion of the tubular body, the torsion spring comprising a first portion attached to the tubular body, a second portion that is radially movable relative to the tubular body, and a locking element on the second portion; and
the outer locking member is sized to engage the locking element.

22. The endoscopic medical device of claim 21, wherein:
the torsion spring is formed from a sheet material that bends around the tubular body; and
the locking element is a rib on the movable portion of the torsion spring.

23. The endoscopic medical device of claim 21, wherein:
the torsion spring is formed from a wire that bends around the tubular body, the wire comprising a locking segment; and
the locking element is the locking segment of the wire.

24. The endoscopic medical device of claim 23, wherein:
the wire of the inner locking device further comprises a second locking segment; and
the outer locking device comprises a second outer locking member sized to engage the second locking segment of the wire.

25. The endoscopic medical device of claim 21, wherein:
the inner locking device includes a plurality of torsion springs formed from separate wires, each wire bent around the tubular body and comprising a locking segment; and
the outer locking device includes a plurality of outer locking members, each of the outer locking members sized to engage at least one of the locking segments.

26. An endoscopic system comprising:
an endoscope having a longitudinal channel;
an endoscopic medical device including a tubular body, wherein the endoscopic medical device is disposed in the longitudinal channel; and
a locking assembly that interlocks the endoscope and the endoscopic medical device to prevent relative rotational movement between the endoscope and the endoscopic medical device.

27. The system of claim 26, wherein the locking assembly includes a locking device attached to the tubular body and a cap attached to the endoscope, wherein the cap is selectively engaged with the locking device.

28. The system of claim 27, wherein:
the cap comprises a side wall and an end wall attached to the side wall at an angle; and
an opening is formed through the end wall and is sized to allow the tubular body to pass through the cap.

29. The system of claim 28, wherein:
the end wall has a hole for an imaging device and a hole for a light source; and
the outer locking device further comprises a polarizing filter disposed over either one or both of the hole for an imaging device and the hole for a light source.

30. The system of claim 28, wherein:
the cap comprises a cylindrical wall extending from the end wall; and
the opening of the outer locking device is formed through the end wall; and
the cylindrical wall is concentric with the opening.

31. The system of claim 30, wherein the outer locking device comprises at least one rib disposed on an inner surface of the cylindrical wall.

32. The system of claim 30, wherein the cylindrical wall has at least one notch formed into an inner surface of the cylindrical wall.

33. The system of claim 27, wherein:
the locking device comprises a flexible flap having a first end attached to the tubular body and a second end that is radially movable relative to the tubular body; and
the cap comprises engages the second end of the flap to prevent the tubular body from rotating relative to the cap.

34. The system of claim 27, wherein:
the locking device comprises a wire spring comprising a medial portion and at least one end attached to the tubular body, the medial portion being radially movable relative to the tubular body; and
the cap selectively engages the medial portion of the wire spring to prevent the tubular body from rotating relative to the cap.

35. The system of claim 34, wherein the medial portion of the wire spring is longitudinally oriented.

36. The system of claim 34, wherein the medial portion of the wire spring is disposed further away from a central longitudinal axis of the tubular body than the rest of the wire spring.

37. The system of claim 34, wherein:
the locking device further comprises a second wire spring comprising a medial portion and at least one end attached to the tubular body, the medial portion of the second wire spring being radially movable relative to the tubular body; and
the cap selectively engages the medial portion of the second wire spring to prevent the tubular body from rotating relative to the cap.

38. The system of claim 34, further comprising position indicator attached to the tubular body, the position indicator being longitudinally aligned with the locking device.

39. The system of claim 38, wherein the position indicator comprises a distal portion, a proximal portion, and at least one end attached to the tubular body, the distal portion oriented at a first acute angle relative to the longitudinal axis, the proximal portion oriented at a second acute angle relative to the longitudinal axis.

40. The system of claim 39, wherein the first acute angle is less than the second acute angle.

41. The system of claim 34, wherein:
the medial portion of the wire spring comprises a curved segment that has a peak disposed further away from the central longitudinal axis of the tubular body than the rest of the wire spring; and
the cap selectively engages the peak to prevent the tubular body from rotating relative to the cap.

42. The system of claim 34, wherein:
the medial portion of the wire spring comprises a distal segment oriented at a first acute angle to the longitudinal axis, a proximal segment oriented at a second acute angle to the longitudinal axis, and an intermediate segment disposed between the distal segment and the proximal segment; and
the cap selectively engages the intermediate segment to prevent the tubular body from rotating relative to the cap.

43. The system of claim 27, wherein:
the locking device comprises a leaf spring comprising at least one end attached to the tubular body, the leaf spring being radially movable relative to the tubular body; and
the cap selectively engages the leaf spring to prevent the tubular body from rotating relative to the cap.

44. The system of claim 43, wherein:
the leaf spring comprises a distal portion oriented at a first angle to the longitudinal axis, a proximal portion oriented at a second angle to the longitudinal axis, and an intermediate section oriented longitudinally and disposed between the distal portion and the proximal portion; and
the cap selectively engages the intermediate section to prevent the tubular body from rotating relative to the cap.

45. The system of claim 43, wherein the locking device further comprises a second leaf spring comprising at least one end attached to the tubular body, the second leaf spring being radially movable relative to the tubular body; and
the cap selectively engages the second leaf spring to prevent the tubular body from rotating relative to the cap.

46. The system of claim 27, wherein:
the locking device comprises a torsion spring disposed circumferentially around a portion of the tubular body, the torsion spring comprising a first portion attached to the tubular body, a second portion that is radially movable relative to the tubular body, and a locking element on the movable portion; and
the cap selectively engages the locking element to prevent the tubular body from rotating relative to the cap.

47. The system of claim 46, wherein:
the torsion spring is formed from a sheet material that bends around the tubular body; and
the locking element is a rib on the movable portion of the torsion spring.

48. The system of claim 46, wherein:
the torsion spring is formed from a wire that bends around the tubular body, the wire comprising a locking segment; and
the locking element is the locking segment of the wire.

49. The system of claim 48, wherein:
the wire of the locking device further comprises a second locking segment; and
the cap engages the second locking segment to prevent the tubular body from rotating relative to the cap.

50. The system of claim 46, wherein:
the locking device includes a plurality of torsion springs formed from separate wires, each wire bent around the tubular body and comprising a locking segment; and
the cap engages the at least one of the locking segments to prevent the tubular body from rotating relative to the cap.

51. A method of configuring an endoscopic system, comprising:
inserting a tubular body of an endoscopic medical device into a channel of an endoscope, wherein an inner locking device is attached to the tubular body, and wherein when the tubular body is inside the channel the inner surface of the channel radially compresses the inner locking device;
continuing to insert the tubular body of the endoscopic medical device until the inner locking device comes out of the channel, wherein the inner locking device extends radially outwards to engage an outer locking device attached to the endoscope, and wherein the engagement prevents the tubular body from rotating relative to the endoscope.
